(19) United States
(12) Patent Application Publication
(10) Pub. No.: US 2018/0338905 A1
(43) Pub. Date: Nov. 29, 2018

(54) BIOAVAILABLE AEROSOLIZED SUPPLEMENT FORMULATIONS

(71) Applicant: Inhale Health, LLC, Los Angeles, CA (US)

(72) Inventors: Daniel Frederick Woolf Shapiro, Los Angeles, CA (US); Mario Danek, Los Angeles, CA (US); Joseph G. Walsh, Los Angeles, CA (US)

(21) Appl. No.: 15/606,546

(22) Filed: May 26, 2017

(51) Int. Cl. A61K 9/00; A61K 31/465; A61K 47/10; A61K 31/714; A61K 31/51; A61K 31/525; A61K 31/4415; A61K 31/375; A61K 31/4188; A61K 31/4045; A61K 31/197; A61K 31/522; A61M 11/04; A61M 15/06; A24F 47/00

(52) U.S. Cl. CPC A61K 9/007; H01M 10/0525; A61K 47/10; A61K 31/714; A61K 31/51; A61K 31/525; A61K 31/4415; A61K 31/375; A61K 31/4188; A61K 31/4045; A61K 31/197; A61K 31/522; A61M 11/042; A61M 15/06; A24F 47/008; H01M 2220/30; A61K 31/465

(57) ABSTRACT

Methods of preparing inhalable supplements are disclosed. The methods involve aerosolizing a liquid comprising a carrier and a supplement by heating the liquid to a temperature that retains the bioavailability of the supplement.

### BACKGROUND

Dietary supplements provide supplemental nutrition and health benefits to subjects in need. Various vitamins, for example, are essential nutrients that contribute to a subject's health. Vitamin B12 (cobalamin), for example, contributes to proper red blood cell formation, neurological function, and DNA synthesis, and vitamin B6 (pyridoxine) is involved in neurotransmitter biosynthesis, maintaining normal levels of homocysteine, and maintaining immune function. In addition, vitamin B1 (thiamine) is involved in energy metabolism and the growth, development, and function of cells. Further, vitamin B2 (riboflavin) is an essential component of the coenzymes flavin mononucleotide and flavin adenine dinucleotide (FAD), which are involved in energy production; cellular function, growth, and development; and metabolism of fats, drugs, and steroids. Consumption of supplements such as vitamins in pill format, however, can be highly inefficient due to low oral absorption rates.

### SUMMARY

The disclosure provides a method of preparing an inhalable supplement comprising aerosolizing a liquid comprising a carrier and an inhalable supplement by heating the liquid to a temperature of about 25° C. to about 300° C. using an atomizer, thereby providing an inhalable vapor containing the supplement.

The disclosure also provides a method of preparing an inhalable supplement comprising aerosolizing a liquid comprising a carrier and an inhalable supplement by heating the liquid for 1 to 10 seconds using an atomizer, wherein (i) the atomizer comprises a heating element connected to a battery; (ii) the heating element has a resistance of about 0.4 ohms to about 6 ohms; and (iii) the battery has a voltage of 1- to 9.1-volts; thereby providing an inhalable vapor containing the inhalable supplement. In some cases, the liquid is provided to the heating element using a wicking material to draw the liquid to the heating element.

The disclosure further provides a method of preparing an inhalable supplement comprising aerosolizing a liquid comprising a carrier and an inhalable supplement by heating the liquid for 1 to 10 seconds using an atomizer, wherein (i) the atomizer comprises a heating element connected to a battery; and (ii) the power generated by the battery connected to the heating element is about 0.2 to about 200 Joules/second; thereby providing an inhalable vapor containing the supplement. In some cases, the liquid is provided to the heating element using a wicking material to draw the liquid to the heating element.

The disclosure also provides a method of preparing an inhalable supplement comprising: aerosolizing a liquid comprising a carrier and an inhalable supplement by heating the liquid for 1 to 10 seconds using an atomizer, wherein (i) the atomizer comprises a heating element connected to a battery; (ii) the heating element has a resistance of about 0.4 ohms to about 6 ohms; (iii) the battery has a voltage of 1- to 9.1-volts; and (iv) the liquid has a mass of about 0.1 mg to about 100 g; thereby providing an inhalable vapor containing the supplement.

The disclosure further provides a method of preparing an inhalable supplement comprising: aerosolizing a liquid comprising a carrier and an inhalable supplement by heating the liquid for 1 to 10 seconds using an atomizer, wherein (i) the atomizer comprises a heating element connected to a battery; (ii) the power generated by the battery connected to the heating element is about 0.2 to about 200 Joules/second; and (iii) the liquid has a mass of about 0.1 mg to about 100 g; thereby providing an inhalable vapor containing the supplement.

Additional features and advantages of the disclosed formulations and methods are described in, and will be apparent from, the following Detailed Description.

Those skilled in the art will readily understand that all embodiments disclosed in the following are examples of specific embodiments, but are not necessarily limiting the general inventive concept. Furthermore, all exemplary embodiments can be read on all inventive aspects and embodiments in any combination, if not mutually exclusive. All equivalents or obvious alterations or modifications as recognized by those skilled in the art are included by the present invention.

### DETAILED DESCRIPTION

The disclosure is directed to methods of preparing inhalable supplements and to related supplement preparations. The method comprises aerosolizing a liquid comprising a carrier and an inhalable supplement by heating the liquid using an atomizer, thereby providing an inhalable vapor containing the inhalable supplement. The disclosed methods advantageously aerosolize the inhalable supplement so as to provide a bioavailable supplement having a high pulmonary absorption rate.

In an embodiment, the liquid comprising the carrier and the inhalable supplement is present as a mixture, for example, a homogeneous mixture or a heterogeneous mixture. In an embodiment, the liquid comprising the carrier and the inhalable supplement is present as a suspension. In an embodiment, the liquid comprising the carrier and the inhalable supplement is present as a solution. In various embodiments, the liquid is free of nicotine. In various other embodiments, the liquid comprises nicotine.

In an embodiment, the liquid comprises about 51% (w/w) to about 99.99999% (w/w) carrier. In an embodiment, the liquid comprises about 0.00001% (w/w) to about 49% (w/w) inhalable supplement.

Suitable carriers include, but are not limited to glycerin, such as vegetable glycerin, 1,2-propanediol, 1,3-propanediol, and combinations thereof. In an embodiment, the carrier comprises a mixture of glycerin, 1,2-propanediol, and 1,3-propanediol. In an embodiment, the carrier comprises a mixture of glycerin and 1,3-propanediol. In an embodiment, the carrier comprises a mixture of glycerin and 1,2-propanediol. In an embodiment, the carrier comprises a mixture of 1,2-propanediol and 1,3-propanediol. In some embodiments, the carrier comprises glycerin and 1,3-propanediol present in a weight ratio of 1:1000 to 1000:1. In some embodiments, the carrier comprises glycerin and 1,2-propanediol present in a weight ratio of 1:1000 to 1000:1.

Suitable supplements include, but are not limited to, vitamin B1, vitamin B2, vitamin B6, vitamin B12, ascorbic acid, biotin, melatonin, L-theanine, caffeine, and combinations thereof. In an embodiment, the inhalable supplement comprises vitamin B1, vitamin B2, vitamin B6, vitamin B12, ascorbic acid, biotin, or combinations thereof. In an embodiment, the inhalable supplement comprises vitamin B12. In an embodiment, the inhalable supplement comprises a mixture of vitamin B1, vitamin B2, vitamin B6, and vitamin B12. In an embodiment, the inhalable supplement comprises ascorbic acid. In an embodiment, the inhalable supplement comprises biotin. In an embodiment, the inhalable supplement comprises melatonin. In an embodiment, the inhalable supplement comprises L-theanine. In an embodiment, the inhalable supplement comprises caffeine. In an embodiment, the inhalable supplement comprises a mixture of L-theanine and caffeine.

In an embodiment, the inhalable supplement has a boiling point greater than the temperature to which the liquid is heated.

In an embodiment, the liquid is heated for about 1 to about 10 seconds.

In an embodiment, the atomizer comprises a heating element to vaporize the liquid. In some cases, the atomizer further comprises a delivery mechanism that actively or passively transports the liquid to the heating element. In some cases, the atomizer further comprises a wicking material as a delivery mechanism to draw the liquid to the heating element. In some cases, the atomizer does not contain a wicking material. In some cases, the liquid is dripped directly onto the heating element. In some cases, the atomizer is a ceramic atomizer. In an embodiment, the heating element has a resistance of about 0.4 ohms to about 6 ohms. In an embodiment, the heating element is connected to a 1- to 9.1-volt battery.

In an embodiment, the vapor produced by the method described herein contains particles having a mass mean aerodynamic diameter of less than about 50 microns.

In an embodiment, inhalation of the vapor by a subject increases plasma levels of the supplement in the subject. In an embodiment, plasma levels of the supplement about 1 to 120 minutes, for example, about 60 to about 90 minutes after inhalation of the vapor are detectably increased compared to plasma levels before inhalation of the vapor, for example, blood plasma levels of the supplement are increased by at least 3%.

### Formulations

The liquid preparations disclosed herein include a carrier component and an inhalable supplement component. The liquid preparations are present in a device that is suitable to aerosolize the liquid, thereby providing an inhalable vapor containing the supplement. Typically, about 0.1 to about 100 mL of liquid is present in the device, for example, about 0.1 to about 10 mL, about 0.5 to about 2 mL, about 1 to 1.5 mL, and/or about 1 to about 1.2 mL. In some cases, about 0.1 mg to about 100 g of liquid is present in the device, for example, about 500 mg to about 1.5 g, about 200 mg to about 1.2 g, about 0.1 mg to about 15 mg, about 0.2 mg to about 10 mg, about 0.5 mg to about 5 mg, about 0.5 mg to about 3 mg, about 1 mg to about 2 mg, about 1.1 mg to about 1.7 mg, and/or about 1.3 mg to about 1.5 mg of liquid. The liquid comprising the carrier and the inhalable supplement can be present as a mixture, for example, a homogeneous mixture or a heterogeneous mixture, a solution, or a suspension.

The carrier component includes any carrier suitable for aerosolizing the inhalable supplement at relatively low temperatures, such as at a temperature below the boiling point of the inhalable supplement. Without wishing to be bound by theory, it is believed that aerosolizing the inhalable supplement at a temperature below its boiling point reduces degradation of the inhalable supplement, thereby facilitating delivery of the bioavailable inhalable supplement to a subject in an amount sufficient to achieve a desired effect in the subject. The liquid preparations contain an amount of carrier suitable to allow a desired amount of inhalable supplement to be aerosolized under conditions that allow the bioactive supplement to be delivered to a subject. In some cases, the liquid contains the carrier component and the inhalable supplement in a weight ratio of about 1:1 to about 1000:1, about 2:1 to about 1000:1, about 5:1 to about 1000:1, about 10:1 to about 1000:1, about 5:1 to about 10:1, about 7:1 to about 10:1, and/or about 1:1 to about 1:1000. Suitable carriers include, but are not limited to glycerin, such as vegetable glycerin, 1,2-propanediol, 1,3-propanediol, and combinations thereof. In some cases, the carrier is a mixture of glycerin, 1,2-propanediol, and 1,3-propanediol. In some cases, the carrier is a mixture of glycerin and 1,3-propanediol. In some cases, the carrier is a mixture of glycerin and 1,2-propanediol. In some cases, the carrier is a mixture of 1,2-propanediol and 1,3-propanediol. In some cases, the carrier comprises glycerin and 1,3-propanediol in a weight ratio of 1:1000 to 1000:1, for example, 1:10 to 10:1, 1:7.5 to 7.5:1, 1:5 to 5:1, 1:4 to 4:1, 1:3 to 3:1, 1:2 to 2:1, 1:1.5 to 1.5:1, and/or 1:1. In some cases, the carrier comprises glycerin and 1,2-propanediol in a weight ratio of 1:1000 to 1000:1, for example, 1:10 to 10:1, 1:7.5 to 7.5:1, 1:5 to 5:1, 1:4 to 4:1, 1:3 to 3:1, 1:2 to 2:1, 1:1.5 to 1.5:1, and/or 1:1.

The carrier component often comprises more than half of the total weight of the liquid. In some cases, for example, the liquid comprises about 51% (w/w) to about 99.99999% (w/w), about 60% (w/w) to about 99.9999%, about 60% (w/w) to about 99.999%, about 60% (w/w) to about 99.99%, about 60% (w/w) to about 99.9%, about 60% (w/w) to about 95% (w/w), about 70% (w/w) to about 95% (w/w), about 80% (w/w) to about 99.9% (w/w), about 80% (w/w) to about 95% (w/w), about 80% (w/w) to about 90% (w/w), and/or about 85% (w/w) to about 90% (w/w) carrier based on the total weight of the liquid. In such cases, the supplement comprises less than half of the total weight of the liquid. In some cases, for example, the liquid comprises about 0.00001% (w/w) to about 49% (w/w), about 0.0001% (w/w) to about 40% (w/w), about 0.001% (w/w) to about 40% (w/w), about 0.01% (w/w) to about 40% (w/w), about 0.1% (w/w) to about 40% (w/w), about 5% (w/w) to about 40% (w/w), about 5% (w/w) to about 30% (w/w), about 0.01% (w/w) to about 20% (w/w), about 5% (w/w) to about 20% (w/w), about 10% (w/w) to about 20% (w/w), and/or about 10% (w/w) to about 15% (w/w) supplement based on the total weight of the liquid.

In some cases, about 0.1 to about 100 mL of carrier is present in the device, for example, about 0.1 to about 10 mL, about 0.5 to about 2 mL, about 1 to 1.5 mL, and/or about 1 to about 1.2 mL of carrier. In some cases, about 0.1 mg to about 100 g of carrier is present in the device, for example, about 500 mg to about 1.5 g, about 200 mg to about 1.2 g, about 0.1 mg to about 15 mg, about 0.2 mg to about 10 mg, about 0.5 mg to about 5 mg, about 0.5 mg to about 3 mg, about 1 mg to about 2 mg, about 1.1 mg to about 1.7 mg, and/or about 1.3 mg to about 1.5 mg of carrier.

In some cases, about 0.01 mg to about 100 mg of supplement is present in the device, for example, about 0.03 mg to about 1.5 mg, about 0.05 mg to about 1 mg, about 0.1 mg to about 0.5 mg, about 0.15 mg to about 0.2 mg, about 11 mg to about 50 mg, about 43 mg to about 90 mg, about 37 mg to about 100 mg, and/or about 1 mg to about 17 mg of supplement.

Inhalable supplements for use in the formulations and methods disclosed herein include ingredients with biological activity and/or a biological response. In various cases, the inhalable supplements provide a benefit to the health of the subject to which the inhalable supplements are administered. Inhalable supplements include, but are not limited to, dietary supplements, stimulants, relaxants, minerals, vitamins, hormones, and other compounds with biological functionality or a biological response within in the subject. Inhalable supplements include, but are not limited to, nutrients, such as vitamins, minerals, amino acids and proteins, fiber, and fatty acids, and biologically active compounds. Suitable supplements include, but are not limited to, vitamin B1, vitamin B2, vitamin B6, vitamin B12, ascorbic acid, biotin, melatonin, L-theanine, caffeine, and mixtures thereof. In some cases, the supplement comprises a mixture of vitamin B1, vitamin B2, vitamin B6, and vitamin B12. In some cases, the supplement comprises a mixture of L-theanine and caffeine. In some cases, the supplement comprises ascorbic acid. In some cases, the supplement comprises biotin. In some cases, the supplement comprises melatonin. In various cases, the supplement excludes nicotine. Further, in various cases, the liquid is free of nicotine. In some cases, the supplement has a boiling point greater than the temperature to which the liquid is heated. For example, the difference between the temperature to which the liquid is heated and the boiling point of the supplement typically is at least 10° C., for example, at least 20° C., at least 30° C., at least 50° C., at least 75° C., at least 100° C., at least 150° C., and/or at least 200° C.

The liquid formulations may include additional components such as flavorings, crowding reagents, and/or a compound that alters the bioavailability and/or solubility of the inhalable supplement. Suitable flavorings include, but are not limited to French vanilla flavor, cream flavor, regular vanilla flavor, honey, raspberry, strawberry, watermelon, blueberry, peach, cherry, chocolate, dark chocolate, milk chocolate, passion fruit, loganberry, banana, cinnamon, licorice, lychee, dragon fruit, tangerine, banana, tea, milk, lavender, orange, coffee, chamomile, mint, peppermint, spearmint, menthol, and mixtures thereof.

### Methods for Vapor Preparation

The disclosure provides methods for preparing an inhalable vapor containing a supplement. The inhalable vapor is prepared by aerosolizing a liquid preparation containing a supplement as described herein. The liquid preparations are present in a device that is suitable to aerosolize the liquid, thereby providing an inhalable vapor containing the supplement. In particular, the liquid is aerosolized by heating the liquid to a relatively low temperature, such as a temperature below the boiling point of the inhalable supplement. As discussed above, aerosolizing the supplement at a relatively low temperature (i.e., a temperature below the supplement's boiling point) is believed to reduce degradation of the supplement, thereby facilitating delivery of bioactive supplement to a subject. Typically, the liquid is heated to a temperature of about 25° C. to about 300° C., for example, about 25° C. to about 75° C., about 30° C. to about 70° C., about 35° C. to about 105° C., about 100° C. to about 200° C., about 110° C. to about 260° C., and/or about 80° C. to about 300° C.

The liquid generally is heated for a relatively short period of time (e.g., less than 10 seconds) to avoid overheating of the liquid and to rapidly aerosolize the supplement for delivery to the subject. In some cases, the liquid is heated for about 1 to about 10 seconds, for example, about 1 to about 8 seconds, about 1 to about 6 seconds, about 1 to about 5 seconds, about 1 to about 4 seconds, about 1 to about 3 seconds, about 1 to about 2 seconds, about 2 to about 10 seconds, about 2 to about 8 seconds, about 2 to about 6 seconds, about 2 to about 5 seconds, about 2 to about 4 seconds, about 3 to about 10 seconds, about 3 to about 8 seconds, about 3 to about 6 seconds, about 3 to about 5 seconds, about 3 to about 4 seconds, about 1 second, about 2 seconds, and/or about 3 seconds.

Various devices can be used to heat the liquid and aerosolize the supplement. In particular, devices containing an atomizer are used and the atomizer is used to heat the liquid. Exemplary devices contain an atomizer and a power source, such as a battery, connected to the atomizer. In some cases, the atomizer comprises a heating element to vaporize the liquid. In some cases, the atomizer further comprises a delivery mechanism that actively or passively transports the liquid to the heating element. In some cases, the atomizer further comprises a wicking material as a delivery mechanism to draw the liquid to the heating element. In some cases, the atomizer does not contain a wicking material. In some cases, the liquid is dripped directly onto the heating element. In some cases, the atomizer is a ceramic atomizer. In some cases, the heating element has a resistance of about 0.4 ohms to about 6 ohms, for example, about 0.8 ohms to about 5 ohms, about 1 ohm to about 4 ohms, about 1 ohm to about 2 ohms, and/or about 2 ohms to about 3 ohms. Suitable voltages for the battery that is connected to the heating element include, but are not limited to, 1- to 9.1-volts, 3- to 6-volts, 4- to 5-volts, 4- to 4.5-volts, and/or 4.15-volts. In some cases, the power generated by the battery that is connected to the heating element is about 0.2 to about 200 Joules/second, for example, about 0.3 to about 100 Joules/second, about 0.4 to about 75 Joules/second, about 0.5 to about 50 Joules/second, about 1 to about 40 Joules/second, about 2 to about 20 Joules/second, and/or about 3 to about 10 Joules/second. Suitable materials from which the wicking material is comprised include, but are not limited to, cotton, silica, polyester, ceramic, fiberglass, and stainless steel.

The vapor produced by the methods described herein contains particles having a particle size suitable for inhalation and absorption in the lungs of a subject. In some cases, the vapor contains particles having a mass mean aerodynamic diameter of less than about 50 microns for example, about 0.1 micron to about 15 microns, about 1 nm to about 500 nm, about 10 nm to about 250 nm, and/or about 100 nm to about 200 nm.

Inhalation by a subject of a vapor prepared by the methods described herein advantageously increases plasma levels of the supplement in the subject. In some cases, plasma levels of the supplement about 1 to about 120 minutes after inhalation of the supplement (e.g., about 30 to about 90 minutes, about 60 to about 90 minutes, about 70 to 80 minutes and/or about 75 minutes after inhalation) are detectably increased compared to plasma levels before inhalation of the vapor, for example, by at least 3% (e.g., at least 3.5%, at least 4%, at least 4.5%, at least 5%, and/or at least 5.5%).

The present disclosure is further exemplified by the following examples without being limited thereto.

### EXAMPLES

### Example 1: Aerosolization Temperature

The temperature of aerosolization is directly related to the power in watts running through the atomizer. Below 20W, the temperature output of the device is from about 25° C. to about 300° C. with no prior usage.

The power in watts (W) can be obtained from the battery voltage (V) and the heating element resistance (R) based on the relationship W=V²/R. For example, the power for a system having a 4.2 V battery and a resistance of 3 W is 5.9 W (4.2²/3). In another example, the power for a system having a 3.2 V battery and a resistance of 3 W is 3.4 W (3.2²/3). In another example, the power for a system having a 4.2 V battery and a resistance of 1.8 W is 9.8 W (4.2²/1.8).

### Example 2: Vaporization of a Vitamin B12 Formulation.

An electronic cigarette device powered by a 4.2V, 320 mAh lithium-ion battery was used to aerosolize a sample containing vitamin B12 in vegetable glycerin and 1,3-propanediol. The sample was heated to 100 to 260° C. with a 3 ohm heating element in about 2 to about 3 second intervals with about 5 to about 10 second rest periods until a total volume of 100 mg was collected. The mixture contained 60% glycerin and 40% 1,3-propanediol to which 2 mg/ml of vitamin B12 was added. A vacuum was used to draw the vapors through the e-cigarette into an impinger, thereby trapping vaporized vitamin B12 in a water reservoir present in the impinger. Vaporization of vitamin B12 was detected by analyzing the water in the impinger using high-performance liquid chromatography (HPLC).

### Example 3: Vaporization of a Melatonin Formulation

An electronic cigarette device powered by a 4.2V, 320 mAh lithium-ion battery was used to aerosolize a sample containing melatonin in vegetable glycerin and 1,3-propanediol. The sample was heated to 100 to 260 ° C. with a 3 ohm heating element in about 2 to about 3 second intervals with about 5 to about 10 second rest periods until a total volume of 100 mg was collected. The mixture contained 60% glycerin and 40% 1,3-propanediol to which 33 mg/ml melatonin was added. A vacuum was used to draw the vapors through the e-cigarette into an impinger, thereby trapping vaporized melatonin in a water reservoir present in the impinger. Vaporization of melatonin was detected by analyzing the water in the impinger using HPLC.

### Example 4: Vaporization of a L-theanine/Caffeine Formulation

An electronic cigarette device powered by a 4.2V, 320 mAh lithium-ion battery was used to aerosolize a sample containing L-theanine and caffeine in vegetable glycerin and 1,3-propanediol. The sample was heated to 100 to 260° C. with a 3 ohm heating element in about 2 to about 3 second intervals with about 5 to about 10 second rest periods until a total volume of 100 mg was collected. The mixture contained 60% glycerin and 40% 1,3-propanediol to which 7.7 mg/ml L-theanine, 4.6 mg/ml caffeine, and 2 mg/ml vitamin B12 was added. A vacuum was used to draw the vapors through the e-cigarette into an impinger, thereby trapping vaporized L-theanine and caffeine in a water reservoir present in the impinger. Vaporization of L-theanine and caffeine was detected by analyzing the water in the impinger using high-performance liquid chromatography (HPLC).

### Example 5: In Vivo Efficacy of a Vaporized Vitamin Formulation

An electronic cigarette device powered by a 4.2V, 320 mAh lithium-ion battery was used to aerosolize a sample containing vitamin B12 in organic vegetable glycerin and 1,3-propanediol and the aerosolized sample was inhaled by test subjects. The sample was heated to 75 to 260° C. with a 3 Ohm heating element in about 2 to about 3 second intervals with about 5 to about 10 second rest periods for a total of about 90 minutes. The mixture contained 60% glycerin and 40% 1,3-propanediol to which 2 mg/ml vitamin B12 was added. Serum vitamin B12 levels were measured in the test subjects before inhalation of the aerosolized sample and 75 minutes after initial inhalation of the aerosolized sample. Before inhalation, serum B12 levels averaged 403.0 pg/mL. After inhalation, serum B12 levels averaged 424.7 pg/mL, a 5.4% increase compared to B12 levels before inhalation of the sample. This study demonstrated that the aerosolized vitamin B12 rapidly entered the bloodstream when inhaled by the test subjects.

1. A method of preparing an inhalable supplement comprising:
aerosolizing a liquid comprising a carrier and an inhalable supplement by heating the liquid to a temperature of about 25° C. to about 300° C. using an atomizer, thereby providing an inhalable vapor containing the supplement.

2. The method of claim 1, wherein the liquid is a mixture.

3. The method of claim 1, wherein the liquid is free of nicotine.

4. The method of claim 1, wherein the liquid comprises nicotine.

5. The method of claim 1, wherein the liquid comprises about 51% (w/w) to about 99.99999% (w/w) carrier and/or the liquid comprises about 0.00001% (w/w) to about 49% (w/w) inhalable supplement.

6. The method of claim 1, wherein the carrier comprises glycerin, 1,2-propanediol, 1,3-propanediol, or combinations thereof.

7. The method of claim 1, wherein the carrier comprises glycerin and 1,3-propanediol present in a weight ratio of 1:1000 to 1000:1 or glycerin and 1,2-propanediol present in a weight ratio of 1:1000 to 1000:1.

8. The method of claim 1, wherein the supplement comprises vitamin B12.

9. The method of claim 1, wherein the supplement comprises vitamin B1, vitamin B2, vitamin B6, vitamin B12, ascorbic acid, biotin, or combinations thereof.

10. The method of claim 1, wherein the supplement comprises melatonin.

11. The method of claim 1, wherein the supplement comprises L-theanine, caffeine, or a combination thereof.

12. The method of claim 1, wherein the supplement has a boiling point greater than the temperature to which the liquid is heated.

13. The method of claim 1, wherein the liquid is heated for about 1 to about 10 seconds.

14. The method of claim 1, wherein the atomizer comprises a heating element to vaporize the liquid.

15. The method of claim 14, wherein the heating element has a resistance of about 0.4 ohms to about 6 ohms and/or the heating element is connected to a 1- to 9.1-volt battery.

16. The method of claim 1, wherein the vapor contains particles having a mass mean aerodynamic diameter of less than about 50 microns.

17. The method of claim 1, wherein inhalation of the vapor by a subject increases plasma levels of the supplement in the subject.

18. The method of claim 17, wherein plasma levels of the supplement about 60 to about 90 minutes after inhalation of the vapor are increased by at least 3% compared to plasma levels before inhalation of the vapor.

19. A method of preparing an inhalable supplement comprising:
aerosolizing a liquid comprising a carrier and an inhalable supplement by heating the liquid for 1 to 10 seconds using an atomizer, wherein
(i) the atomizer comprises a heating element connected to a battery;
(ii) the heating element has a resistance of about 0.4 ohms to about 6 ohms; and
(iii) the battery has a voltage of 1- to 9.1-volts;
thereby providing an inhalable vapor containing the supplement.

20. A method of preparing an inhalable supplement comprising:
aerosolizing a liquid comprising a carrier and an inhalable supplement by heating the liquid for 1 to 10 seconds using an atomizer, wherein
(i) the atomizer comprises a heating element connected to a battery; and
(ii) the power generated by the battery connected to the heating element is about 0.2 to about 200 Joules/second;
thereby providing an inhalable vapor containing the supplement.
